(19) United States
(12) Patent Application Publication
(10) Pub. No.: US 2018/0200321 A1
(43) Pub. Date: Jul. 19, 2018

(54) COMPOSITIONS COMPRISING PHOENIX DACTYLIFERA AND METHODS OF PREVENTION AND TREATMENT USING THEREOF

(71) Applicant: NADINE NAHEED ABDELHADI, AMMAN (JO)

(72) Inventor: NADINE NAHEED ABDELHADI, AMMAN (JO)

(21) Appl. No.: 15/408,468

(22) Filed: Jan. 18, 2017

(51) Int. Cl. A61K 36/889; A61K 9/06; A61K 9/08; A61K 9/00

(52) U.S. Cl. CPC A61K 36/889; A61K 9/0095; A61K 9/08; A61K 9/06

(57) ABSTRACT

The present invention relates to compositions comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents. The present invention also relates to methods of using said compositions in treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, nutrition disorders, liver diseases, kidney diseases, poisoning, dental problems, infertility, diaper rash, neurological diseases, bone diseases, blood diseases, psychiatric diseases, diabetic foot ulcer, gynecological diseases, premenstrual syndrome, pain management, infections, labor induction and delivery management. The invention further relates to methods of using said compositions as nutritional supplements and cosmoceuticals.

### FIELD OF THE INVENTION

The present invention relates to compositions comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents, and to methods for preparing such compositions. The present invention also relates to methods of using said compositions in treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, liver diseases, kidney diseases, nutrition disorders, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management. The present invention further relates to methods of using said compositions to improve the skin cosmetically and to promote hair growth. The present invention also relates to methods of using said compositions as nutritional supplements.

### BACKGROUND OF THE INVENTION

Phoenix dactylifera is a flowering plant species in the palm family Arecaceae. Its fruits are not only edible, but also tasty.

It is thought to be indigenous to either the Middle East or North Africa. Phoenix dactylifera has been used in the Middle East as the main food source for at least 1000 years.

The Worldwide patent application # WO 2006131932 A1 discloses a herbal formulation capable of preventing alcohol-induced hangover, comprising a safe and effective amount of the extracts and/or powder of Phoenix dactylifera, Cichorium intybus, Andrographis paniculata, Vitis vinifera, Phyllanthus amarus and Emblica officinalis alone or in synergistic composition thereof and an acceptable formulation carrier(s). It also discloses the methods of preparing the said herbal formulation, methods of preparing various dosage forms and methods of preventing hangover effect associated with consumption of alcohol.

The Worldwide patent application # WO 2011114249 A1 discloses a natural substance based antioxidant and chemoprotective agent and its method of production. The natural substance based antioxidant and chemoprotective agent consists of two parts a vegetable component which is prepared from dry extracts from the fruits of Vitis vinifera representing 20 to 60% of weight, from Emblica officinalis 15 to 55% of weight, from Phoenix dactylifera 10 to 55% of weight, from the haulms of Phyllanthus amarus 5 to 20% of weight, Andrographis paniculata from 4 to 20% of weight and from the entire plant of Cichorium intybus or from its parts 7 to 20% of weight and this component is supplemented by one part of the vitamin-mineral component consisting of magnesium of 50 to 70% of weight, zinc 2.8 to 3.4% of weight, molybdenum 0.01 to 0.02% of weight, chromium 0.01 to 0.02% of weight, selenium 0.01 to 0.02% of weight, vitamin C 20 to 30% of weight, vitamin E 3.50 to 4.10% of weight, pyridoxine 0.40 to 0.50% of weight, thiamine 0.30 to 0.40% of weight, riboflavine 0.40 to 0.50% of weight, nicotiamide 5.00 to 5.50% of weight, cyanokobalamin 0.0008 to 0.001% of weight and folic acid 0.06 to 0.07% of weight. The natural substance based antioxidant and chemoprotective agent is produced in such a way that the component is homogenized individually and then mixed in proportion to the vegetable component 2 parts+1 part of the vitamin-mineral component (recalculated into active elements) whereas after the final homogenization, suitable capsules are filled with the solid form of the agent so that the consumer pack does not exceed the daily recommended doses (DRD) or is adjusted in a different suitable solid form while maintaining the same conditions for DRD whereas the liquid form arises after the solid form has been dissolved in the required amount of water so that a solution of 0.15 to 0.3% of weight arises. The liquid form of the agent can contain 0.015 to 0.04% of caffeine weight in the final agent. The agent contains the vegetable component and the vitamin-mineral component in proportion to 2:1 of the weight parts recalculated into efficient elements in the mineral component.

There is a need for novel compositions which are useful for treating and/or preventing a variety of diseases and disorders, and also for new methods of treatment using said novel compositions.

### SUMMARY OF THE INVENTION

The present invention relates to compositions comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents, and to methods for preparing such compositions.

The present invention also relates to methods of using said compositions in treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, liver diseases, kidney diseases, nutrition disorders, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management.

The present invention further relates to methods of using said compositions to improve the skin cosmetically and to promote hair growth.

The present invention also relates to methods of using composition as nutritional supplements.

In an embodiment of the present invention, the invention relates to a method for treatment and prevention of neonatal hyper bilirubinemia by oral or enteral administration of a Phoenix dactylifera preparation to treat and prevent neonatal hyperbilirubinemia. A composition that can be used comprises Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents.

The present invention further provides a method for preparing said compositions; the method comprises the following steps:

1. Washing Phoenix dactylifera fruit thoroughly with purified water.

2. Soaking Phoenix dactylifera fruit in cold water.

3. Remove seeds using automatic seeds removing machine.

4. Forming Phoenix dactylifera fruit paste.

5. Forming Phoenix dactylifera fruit extract.

6. Washing Phoenix dactylifera Seeds thoroughly

7. Grinding to form a powder.

8. Forming Phoenix dactylifera seed extract.

9. Preparation of stem cell and stem cell extract.

10. Preparation of nanoparticles.

11. Adding physiologically acceptable additives, carriers or diluents

The steps from 1-11 can be omitted, repeated, rearranged or modified.

The invention also provides a method of treating and/or preventing diseases or conditions, including but not limited to, gastrointestinal disorders, hyperbilirubinemia, skin disorders, wrinkles, scars, wounds, hair disorders, neurological diseases, bone diseases, blood diseases, psychiatric diseases, respiratory diseases, liver diseases, kidney diseases, nutrition disorders, poisoning, dental problems, infertility, diaper rash, neurological diseases, diabetic foot ulcer, gynecological diseases, premenstrual syndrome (PMS), infectious diseases, pain management, induction of labor and delivery management, the method comprises administering a safe and therapeutically effective amount of the said Phoenix dactylifera compositions.

The invention also provides a method of using said compositions to improve the skin cosmetically and to promote hair growth.

The present invention also relates to methods of using composition as nutritional supplements.

In another embodiment of the present invention, the invention also relates to a method for treatment and prevention of diabetic foot ulcer and infections by topical administration of a Phoenix dactylifera preparation to treat and prevent diabetic foot ulcer and infections. A preparation that can be used comprises Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents.

In another embodiment of the present invention, the invention also relates to a method for induction and enhancement of labor by oral or enteral administration of a Phoenix dactylifera preparation to induce and enhance labor. A preparation that can be used comprises Phoenix dactylifera fruit flesh; or Phoenix dactylifera fruit skin; or Phoenix dactylifera fruit extract; or Phoenix dactylifera nanoparticles, or combination thereof as active ingredients and physiologically acceptable additives, carriers or diluents.

### DETAILED DESCRIPTION OF THE INVENTION

The present invention will now be described more fully hereinafter with reference to the examples, which further illustrate the invention described herein. This invention may, however, be embodied in different forms and should not be construed as limited to the embodiments set forth herein. Rather, these embodiments are provided so that this disclosure will be thorough and complete, and will fully convey the scope of the invention to those skilled in the art.

As used herein and in the claims, the singular forms “a,” “an,” and “the” include the plural reference unless the context clearly indicates otherwise.

Unless otherwise defined, all technical and scientific terms used herein have the same meaning as commonly understood by one of ordinary skill in the art to which this invention belongs.

The term “Treatment” as used herein refers to any type of treatment that imparts a benefit to a patient afflicted with a disease, including improvement in the condition of the patient (e.g., in one or more symptoms), delay in the progression of the disease, prevention or delay of the onset of the disease, etc.

The term “Pharmaceutically acceptable” as used herein means that the compound or composition is suitable for administration to a subject to achieve the treatments described herein, without unduly deleterious side effects in light of the severity of the disease and necessity of the treatment.

The term “Therapeutic effective amount” as used herein means the amount required to produce the desired effect.

The present invention relates to compositions comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents, to methods for preparing such compositions.

The present invention also relates to methods of using said compositions in treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, liver diseases, kidney diseases, nutrition disorders, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management.

The present invention further relates to methods of using said compositions to improve the skin cosmetically and promoting hair growth.

The present invention also relates to methods of using composition as nutritional supplements.

In an embodiment of the present invention, the invention also relates to a method for treating and preventing neonatal hyper bilirubinemia by oral or enteral administration of said Phoenix dactylifera composition. A preparation that can be used includes Phoenix dactylifera fruit or extract as active ingredients and physiologically acceptable additives, carriers or diluents.

In another embodiment of the present invention, the invention also relates to a method for treatment and prevention of diabetic foot ulcer and infections by topical administration of a Phoenix dactylifera preparation to treat and prevent diabetic foot ulcer and infections. A preparation that can be used comprises Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents.

In another embodiment of the present invention, the invention also relates to a method for induction and enhancement of labor by oral or enteral administration of a Phoenix dactylifera preparation to induce and enhance labor. A preparation that can be used includes Phoenix dactylifera fruit or extract and/or seeds or extract and/or nanoparticles as active ingredients and physiologically acceptable additives, carriers or diluents.

The present invention further provides a method for preparing said compositions; the method comprises the following steps:

1. Washing Phoenix dactylifera fruit thoroughly with purified water.

2. Soaking Phoenix dactylifera fruit in cold water.

3. Remove seeds using automatic seeds removing machine.

4. Forming Phoenix dactylifera fruit paste.

5. Forming Phoenix dactylifera fruit extract.

6. Washing Phoenix dactylifera Seeds thoroughly

7. Grinding to form a powder.

8. Forming Phoenix dactylifera seed extract.

9. Preparation of stem cell and stem cell extract.

10. Preparation of nanoparticles.

11. Adding physiologically acceptable additives, carriers or diluents

The steps from 1-11 can be omitted, repeated, rearranged or modified.

The present invention is concerned primarily with the treatment of human subjects, but may also be employed for the treatment of other animal subjects (i.e., mammals, avians) for veterinary purposes. Mammals (including but not limited to dogs, cats, rabbits, horses, etc.) are preferred, with humans being particularly preferred.

Compositions of the present invention may be prepared by the process described herein, or variations thereof which will be apparent to those skilled in the art.

The compositions of the present invention are useful as pharmaceutically active agents and may be utilized in bulk form. More preferably, however, these compositions are formulated into pharmaceutical formulations for administration. Any of a number of suitable pharmaceutical formulations may be utilized as a vehicle for the administration of the compositions of the present invention.

The compositions of the present invention may be formulated for administration for the treatment of a variety of conditions.

In the manufacture of a pharmaceutical formulation according to the invention, the compositions of the present invention and the physiologically acceptable salts thereof, or the acid derivatives of either are typically admixed with an acceptable carrier. The carrier must be acceptable in the sense of being compatible with any other ingredients in the formulation and must not be deleterious to the patient. The carrier may be a solid or a liquid, or both, and is preferably formulated with the compound as a unit-dose formulation. One or more of each of the active compositions may be incorporated in the formulations of the invention, which may be prepared by any of the well-known techniques of pharmacy consisting essentially of admixing the components, optionally including one or more accessory ingredients.

The formulations of the invention include those suitable for oral, enteral, rectal, topical, buccal, parenteral, and transdermal administration, although the most suitable route in any given case will depend on the nature and severity of the condition being treated and on the nature of the particular active compound which is being used.

Subjects which may be treated using the compositions of the present invention are typically human subjects although the compositions of the present invention may be useful for veterinary purposes with other subjects, particularly mammalian subjects including, but not limited to, horses, cows, dogs, rabbits, fowl, sheep, and the like. As noted above, the present invention provides pharmaceutical formulations comprising the said compositions, or pharmaceutically acceptable salts thereof, in pharmaceutically acceptable carriers for any suitable route of administration, including but not limited to oral, enteral, rectal, topical, buccal, parenteral, intramuscular, intradermal, intravenous, and transdermal administration.

The therapeutically effective dosage of any specific compound will vary somewhat from compound to compound, patient to patient, and will depend upon the condition of the patient and the route of delivery.

The present invention relates to compositions comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents, and to methods for preparing such compositions. The present invention also relates to a method of using said compositions in treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, nutrition disorders, liver diseases, kidney diseases, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management. The method comprises administering a safe and therapeutically effective amount of the composition containing Phoenix dactylifera.

The invention also provides a method of treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, nutrition disorders, liver diseases, kidney diseases, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management. the method comprises administering a therapeutically effective amount of the composition containing Phoenix dactylifera fruit or extract and/or seed powder or extract and/or stem cells or extract and/or nanoparticles as active ingredients and physiologically acceptable additives, carriers or diluents, the method comprises administering a safe and therapeutically effective amount of the said composition to a subject.

The present invention also relates to methods of using composition as nutritional supplements.

The invention also provides a method of using said compositions to improve the skin cosmetically and promoting hair growth.

In an embodiment of the present invention, the invention also relates to a method for the treatment and prevention of neonatal hyper bilirubinemia by oral or enteral administration of a Phoenix dactylifera preparation to treat and prevent neonatal hyperbilirubinemia. A preparation that can be used comprises Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents

In an another embodiment of the present invention, the invention also relates to a method for treatment and prevention of diabetic foot ulcer and infections by topical administration of a Phoenix dactylifera as active ingredients and physiologically acceptable additives, carriers or diluents composition to treat and prevent diabetic foot ulcer and infections. A composition that can be used comprises Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof and physiologically acceptable additives, carriers or diluents.

In another embodiment of the present invention, the invention also relates to a method for induction and enhancement of labor by oral or enteral administration of a Phoenix dactylifera preparation to induce and enhance labor. A preparation that can be used comprises Phoenix dactylifera fruit flesh; or Phoenix dactylifera fruit skin; or Phoenix dactylifera fruit extract; or Phoenix dactylifera nanoparticles, or combination thereof as active ingredients and physiologically acceptable additives, carriers or diluents.

The present invention is explained in greater detail in the following non-limiting example.

### EXAMPLES

The following examples illustrate the present invention without, however, limiting the same thereto.

### Example 1

Cough: A 34 years old female (60 kg) patient suffered from sore throat and cough. She was given a dose of 0.5 g/kg of Phoenix dactylifera fruit flesh composition according to the present invention (85% of weight). Treatment was given in divided 3 doses. The patient was relieved completely from these symptoms at day 5.

### Example 2

Acne: A 15-year old patient, (55 kg weight) had been bothered by facial acne for the past 2 years. She described facial breakouts that began gradually, varied in severity, worsened during menses, and never completely cleared. She was given a dose of 1 g/kg of the composition of Phoenix dactylifera fruit flesh and skin composition according to the present invention (80% of weight) in 3 divided doses orally. The patient reported marked improvement in her acne after 3 weeks of administration.

### Example 3

Labor induction: A 31 female pregnant (75 kg weight) at 37 week+3 days, At her monthly visit to the gynecologist, she was examined for cervical dilatation and the cervical dilatation was zero. She started taking 1.2 g/kg of the Phoenix dactylifera fruit flesh and skin composition according to the present invention (95% of weight) in 3 divided doses orally at week 37+3 days and after three days of consumption cervical dilatation was 5 cm and was admitted to hospital for delivery. She reported less pain than prior delivery and labor time was much shorter.

### Example 4

Wrinkle: A 60 years old woman used 5% cream of the Phoenix dactylifera fruit flesh composition of the current invention for 4 months. She had Class II wrinkles before initiating therapy. After one month of treatment she reported marked improvement in wrinkle appearance.

### Example 5

Neonatal hyperbilirubinemia: 5 days old male newborn (3.25 kg weight) total serum bilirubin level was 16 mg per dL. The newborn was given 0.05 g/kg of the Phoenix dactylifera fruit flesh and skin composition according to the present invention (70% of weight) as oral drops in 3 divided doses. At day 2 total serum bilirubin level dropped to 12 mg per dL. Total serum bilirubin level was 7 mg per dL after one week. Treatment was discontinued after 8 days due to normalization of total serum bilirubin level.

### Example 6

Premenstrual syndrome (PMS): Cough: A 34 year's old female (60 kg) patient suffering from moderate PMS. She was given a dose of 0.5 g/kg of Phoenix dactylifera orally fruit flesh composition according to the present invention (95% of weight) for 2 days prior to menstruation. Treatment was given in divided 3 doses. The patient reported a marked improvement in PMS mood swings, anxiety, and breast tenderness and menstruation pain.

### Example 7: Biological Activity

Antimicrobial assay was performed according to the EUCAST standards of disk diffusion antimicrobial susceptibility test.

The 90% of weight of Phoenix dactylifera fruit flesh and skin composition showed a significant antimicrobial activity against Staphylococcus aureus, the inhibition zone was found to be 7 mm in diameter.

The 90% of weight of Phoenix dactylifera seed also showed a significant antimicrobial activity against Staphylococcus aureus, the inhibition zone was found to be 9 mm in diameter.

Moreover, said compositions was also tested against MRSA S. aureus and the inhibition zone was found to be 12 mm, 13 mm respectively against MRSA

Antifungal Screening of the said compositions showed some antifungal activity against some isolates of yeasts and molds.

While the present invention has been described in details and with reference to specific embodiments thereof, it will be apparent to one skilled in the art that various additions, omissions and modifications can be made without departing from the spirit and scope thereof.

1. A composition for treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, nutrition disorders, liver diseases, kidney diseases, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management comprising: Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents.

2. The composition of claim 1 wherein the active ingredients of the composition comprise in weight percent:
Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof: 0.01-95%,

3. A method for treating and/or preventing diseases or conditions, including but not limited to, gastrointestinal disorders, hyperbilirubinemia, skin disorders, wrinkles, scars, wounds, hair disorders, oncological diseases, endocrinological disorders, immune disorders, respiratory diseases, nutrition disorders, liver diseases, kidney diseases, poisoning, dental problems, infertility, diaper rash, neurological diseases, bone diseases, blood diseases, psychiatric diseases, diabetic foot ulcer, gynecological diseases, premenstrual syndrome (PMS), pain management, infections, induction of labor and delivery management by administering to a subject a safe and therapeutically effective amount of a composition consisting essentially of Phoenix dactylifera fruit flesh; or Phoenix dactylifera fruit skin; or Phoenix dactylifera fruit extract; or Phoenix dactylifera seed extract; or Phoenix dactylifera stem cells extract; or Phoenix dactylifera nanoparticles, or combination thereof as active ingredients and physiologically acceptable additives, carriers or diluents.

4. A method for treating and/or preventing neonatal hyperbilirubinemia by administering to an infant a safe and therapeutically effective amount of an antihyperbilirubinemia composition consisting essentially of Phoenix dactylifera fruit flesh; or Phoenix dactylifera fruit skin; or Phoenix dactylifera fruit extract, or combination thereof as active ingredients and physiologically acceptable additives, carriers or diluents.

5. The method of claim 4 wherein the preparation is administered to newborn infant.

6. The method of claim 4 wherein the preparation is in gel form.

7. The method of claim 4 wherein the preparation is in solution form.

8. The method of claim 4 wherein the preparation is in suspension form.

9. The method of claim 4 wherein the preparation is administered orally or enterally.

10. The method of claim 4 wherein the preparation is administered with breast milk, humanized milk or infant formula.
